(19) United States
(12) Patent Application Publication
(10) Pub. No.: US 2023/0231330 A1
(43) Pub. Date: Jul. 20, 2023

(54) SPRING LOCKING ELECTRODE CONNECTOR APPARATUS WITH MULTICONDUCTIVE CONTACTS AND METHODS THEREOF

(71) Applicant: ADVANTAGE MEDICAL ELECTRONICS, LLC, CORAL SPRINGS, FL (US)

(72) Inventor: IAN WEST, PARKLAND, FL (US)

(73) Assignee: ADVANTAGE MEDICAL ELECTRONICS, LLC, CORAL SPRINGS, FL (US)

(21) Appl. No.: 18/156,948

(22) Filed: Jan. 19, 2023

Related U.S. Application Data
(60) Provisional application No. 63/300,823, filed on Jan. 19, 2022.

(51) Int. Cl. H01R 11/24; A61B 5/273; A61B 5/28

(52) U.S. Cl. CPC H01R 11/24; A61B 5/273; A61B 5/28; H01R 2201/12; A61B 2562/227

(57) ABSTRACT

A novel ECG electrode connector adapted for attachment to a biomedical patient electrode by either pinch or snap connection is disclosed. A closed-end electrical connector includes a pair of pivotally connected members including a main connector body having two electrically conductive contacts located in proximity to one side of an ECG stud, and a jaw pivotally connected thereto and resiliently biased to a closed position. An ECG electrode connector in accordance with the present disclosure may further be fabricated of radiolucent materials.

### CROSS REFERENCE TO RELATED APPLICATIONS

This application claims priority to U.S. Provisional Patent Application No. 63/300,823 filed Jan. 19, 2022, entitled SPRING LOCKING ELECTRODE CONNECTOR APPARATUS WITH MULTICONDUCTIVE CONTACTS AND METHODS THEREOF, which is hereby incorporated by reference in its entirety as though fully set forth herein.

### TECHNICAL FIELD

The present disclosure relates generally to electrode connectors and methods of using electrode connectors, and more particularly, the present disclosure relates to electrocardiograph systems in which electrode connectors are utilized for electrically connecting a signal-conducting leadwire from an electrocardiograph patient monitoring system to a biomedical electrode affixed to a patient.

The present disclosure relates to the field of electrode connectors for attachment to a biomedical patient electrode using a closed-end electrode connector comprising a main connector body and a manually actuated jaw member. The electrical connector is configured in a normally closed position, but can be adjusted to an opened position by biasing the manually actuated jaw member towards the main connector body at which time the electrode connector can be attached to the patient electrode.

The present disclosure relates to the field of electrode connectors in which the main body comprises an opening to receive the stud of a biomedical electrode therein. The jaw member in the preferred embodiment comprises an arc-shaped electrically conductive side contact and, in an alternative embodiment, a plane-shaped electrically conductive top contact, both electrically connected to a leadwire at one end, and making optimal electrical contact with the biomedical electrode stud when the manually actuated lever is biased to the closed position.

The present disclosure relates to the field of electrode connectors in which the novel ECG electrode connector in an alternative embodiment is configured to be manufactured using radiolucent materials such that the electrode connector has radiolucent characteristics.

### BACKGROUND OF DISCLOSURE

When an individual is healthy, his or her heart performs an orderly sequence of steps that give rise to normal features or characteristics. When an individual is not healthy, however, those characteristics may be altered. By tracking and recording the electrical activity of the heart, one can potentially determining the cause or reason for the individual's health issues.

To observe, measure and record the heart's electrical activity, an electrocardiogram (ECG or EKG) system is commonly used. An ECG system is a diagnostic tool that produces an electrocardiograph or a graphical representation of that electrical activity. The electrocardiograph records the electrical voltage in the heart in the form of a continuous strip graph, which can be useful for screening and detecting cardiovascular diseases.

To the trained eye, the ECG conveys considerable information about the structure of the heart and the function of its electrical conduction system. These electrocardiograph records are the main tool in cardiac electrophysiology. Interpretation of these details allows diagnosis of a wide range of heart conditions. Among other things, an ECG can be used to measure the rate and rhythm of heartbeats, the effects of heart drugs, and the function of implanted pacemakers.

Additionally, the ECG can assist in determining whether the heart is performing normally or suffering from abnormalities, while also indicating acute or previous damage to the heart muscle from a heart attack, from an inadequate blood supply to the heart muscles, such as angina, or from conduction abnormalities, such as heart or branch blockage. The ECG can also be used to detect potassium, calcium, magnesium and other electrolyte disturbances and can be used as a screening tool for ischaemic heart disease during an exercise tolerance test. Along those lines, the ECG can be utilized to provide information on the physical condition of the heart during stress testing, and be used to diagnose other, non-cardiac diseases.

In general, an ECG is constructed by measuring electrical potential between various points of the body using a high impedance differential amplifier or some other measuring device along with electrodes attached to locations on the patient's body.

In one exemplary embodiment, leads are placed for measurement over the limbs from the right to the left arm, from the right arm to the left leg and from the left arm to the left leg, creating an imaginary point located centrally in the chest above the heart. Other measurements are derived from potential between this chest location and the three limb leads and six precordial leads, resulting in as many as twelve leads in total.

In general, the ECG system relies typically on electrodes placed on a patient in these and other specific locations to detect the electrical impulses generated by the heart during each beat. These electrical impulses are detected by the electrodes and are communicated to an ECG monitor through a plurality of leadwires, each of which terminate with an electrically conductive electrode connector that is physically connected to one of the electrodes, thereby creating an electrical connection from the patient to the monitor. Since these electrical signals are weak, typically from 0.5 mV to 2.0 mV, each aspect of the system must perform well, in other words, must be able to acquire and transmit the signal without too much signal loss as it propagates over the system, be less sensitive to motion induced noise, etc. to accurately capture the vital information.

Electrode connectors can be attached to the studs of biomedical patient electrodes, which are affixed to the patient, in one of two ways, by using a pinch connection device or by using a snap or press connection device.

Examples of a connector electrode are disclosed in U.S. Pat. Nos. 9,226,680 and 10,010,257, issued to Kendricks, which disclose improved ECG electrode connectors adapted for attachment to a biomedical patient electrode by either pinch or snap connection. Closed-end electrical connectors include a pair of pivotally connected members including a main connector body having an electrically conductive plate defining an electrode stud receiving aperture and disposed in proximity to the bottom surface thereof, and a jaw pivotally connected thereto and resiliently biased to a closed position. The jaw is adapted with a beveled surface that functions to urge the jaw open by engagement of the top surface of an ECG stud thereby allowing the connector to be attached by snap engagement. The electrically conductive plate defines an irregular, generally oval-shaped opening that allows the electrode stud to be inserted through a wide portion of the opening and retained by the narrow portion of the opening. Positioning an electrically conductive plate at the bottom of the connector allows the connector to maximize electrical contact with the electrode stud. ECG electrode connectors in accordance with the present disclosure may further be fabricated of radiolucent materials.

Additional examples of ECG electrode connectors are disclosed in U.S. Pat. No. 8,038,484 and in various continuation thereof, issued to Selvitelli et al., which disclose ECG electrode lead wire connectors which provide improved electrical and mechanical coupling of the ECG electrode press stud to the lead wire, provide enhanced ergonomics to the clinician, and may alleviate patient discomfort associated with the attachment and removal of ECG leads. The connectors may be engaged and disengaged with little or no force imparted to the patient or the ECG pad, which significantly minimizes the risk of inadvertent dislodgement of the pad. In one embodiment, the disclosed connectors provide a thumb cam lever, which affirmatively engages the press stud to the connector, and provides tactile feedback to the clinician that the connectors are properly engaged. In other embodiments, the connectors provide a pushbutton to enable the clinician to easily engage and disengage the connectors from the ECG stud. The disclosed connectors may also decrease clinician fatigue, and may provide more reliable ECG results.

Another example of a pinch connector is disclosed in U.S. Pat. No. 4,178,052, issued to Ekbom, which discloses a medical terminal clip that has a body member with a longitudinal axis and a pair of laterally spaced leg members extending in approximately the longitudinal direction and pivotally connected for relative movement. The respective spaced leg members form a variably spaced electrode receptacle on one side of the pivotal connection. A beryllium copper conductive member is embedded in the body member and is formed from a strip of metal bent into approximately an M-shape with side flanges on the leg members to provide additional strength. A shield or barrier member extends at least between the approximate ends of the leg members on the other side of the pivotal connection while permitting relative movement of the leg members. The shield member is designed to close longitudinal access to the space between the leg members and thereby prevent any dislocation of the terminal clip member by catching onto exterior objects such as other terminal wires.

Among other disadvantages, open ended connectors are burdened with a tendency to snare or snag leadwires that happen to come between the openings between the jaws. When wires become snagged, the connector or the entire connector and electrode assembly can be accidentally dislodged from the patient.

To obviate this problem, another category of electrode connectors is referred to as the “closed end” electrode connectors. As an example, U.S. Pat. No. 4,390,223, issued to Zenkich, discloses an electrical connector for a cable and an electrode assembly having a resilient electrical conducting wire with two ends and an expandable polygonal loop therebetween with the ends of the conducting wire embedded in a handle assembly composed of two handles with a loop circumscribing hinge therebetween. An electrically conducting cable is connected with one of the conductor wire ends within one of the handles and has a plug on the other end for insertion into a monitoring instrument jack. The connection of the cable to the electrode is accomplished by movement of the handles of the electrical connector toward one another, which expands the loop so as to fit over the electrode and release of the handles causing the loop to contract and engage the electrode.

Depending on the healthcare environment, pinch connectors, open ended or closed ended may be preferred. Either way, there continues to exist a need for improvements in the area of electrode connectors for ECG and EKG systems that maximizes the advantages of pinch connectors, while minimizing or eliminating the disadvantages of the existing electrode connectors.

### SUMMARY OF THE DISCLOSURE

The present disclosure relates generally to electrode connectors and methods of using electrode connectors for patient monitoring and reporting. More particularly, the present disclosure relates to electrocardiograph systems in which electrode connectors are utilized for electrically connecting a signal-conducting leadwire from an electrocardiograph patient monitoring system to a biomedical electrode affixed to a patient.

The present disclosure improves on the field of electrode connectors and overcomes the limitations and disadvantages present in the prior art by providing an improved ECG electrode connector adapted for attachment to a biomedical patient electrode or stud using a novel pinch-type connection. In accordance with a preferred embodiment, a closed-end electrical connector comprises a main connector body and a manually actuated jaw member comprising a lever pivotally connected thereto. By placing a force on the lever of the manually actuated jaw member towards the main connector body, the electrode connector can be configured to an open position, however the lever is normally biased to a closed configuration by a biasing member.

The main connector body comprises an opening extending from a top surface to a bottom surface and functions to receive the stud of a biomedical electrode therein. The main connector body of the connector electrode comprises a clamping prong and a bottom surface defining a recessed portion. The jaw member comprises an arc-shaped electrically conductive side contact and a plane-shaped electrically conductive top contact disposed within the jaw member. Both the conductive side contact and the conductive top contact are electrically connected to the leadwires at one end, and are located and configured to make optimal electrical contact with the stud of a biomedical electrode when the manually actuated lever is biased to the closed position.

Positioning both the conductive side contact and the conductive top contact within the jaw member, opposite the clamping prong, allows the connector to maximize electrical contact with the electrode stud by increasing the number of electrical contact points between the electrode connector and the electrode stud when the electrode connector is biased to the closed position. One of the goals of optimized electrical contact is to reduce signal artifact related to patient motion, among others.

The preferred embodiment discloses a pinch clip arrangement, although alternative embodiments may include a beveled lower edge and a snap engagement. Various biasing members and structures are disclosed such that an electrode connector used for ECG monitoring in accordance with the present disclosure may further be fabricated of radiolucent materials.

Accordingly, it is an object of the present disclosure to provide advancements in the field of ECG patient monitoring, by providing an improved electrode connector for establishing optimal electrical communication with an ECG biomedical electrode to facilitate the transmission of signals to ECG monitoring equipment.

In accordance with these and other objects, which will become apparent hereinafter, the instant disclosure will now be described with particular reference to the accompanying drawings.

### DESCRIPTION OF THE DRAWINGS

FIG. 1A shows a top perspective view illustrating an electrode connector in accordance with the present disclosure in relation to a shown biomedical patient electrode;

FIG. 1B shows a top perspective view illustrating an electrode connector in accordance with the present disclosure in relation to a biomedical patient electrode;

FIG. 1C shows a bottom perspective view illustrating an electrode connector in accordance with the present disclosure;

FIG. 2 shows an electrode connector in use attached to a biomedical electrode attached to a patient in accordance with the present disclosure;

FIG. 3A shows an exploded top perspective view of the electrode connector in accordance with the present disclosure;

FIG. 3B shows an exploded top perspective view of the electrode connector in accordance with the present disclosure;

FIG. 3C shows an exploded bottom perspective view of the electrode connector in accordance with the present disclosure;

FIG. 3D shows an exploded bottom perspective view of the electrode connector in accordance with the present disclosure;

FIG. 4A shows exploded side view of an electrode connector in accordance with the present disclosure;

FIG. 4B shows exploded side view of an electrode connector in accordance with the present disclosure;

FIG. 4C shows exploded side view of an electrode connector in accordance with the present disclosure;

FIG. 4D shows exploded side view of an electrode connector in accordance with the present disclosure;

FIG. 5A shows a top view of an electrode connector end view illustrating actuation of the movable jaw in an open position, in accordance with the present disclosure;

FIG. 5B shows a top view of an electrode connector end view illustrating actuation of the movable jaw in a biased opened position, in accordance with the present disclosure;

FIG. 5C shows a top view of an electrode connector end view illustrating actuation of the movable jaw in an closed position, in accordance with the present disclosure;

FIG. 5D shows a top view of an electrode connector end view illustrating actuation of the movable jaw in a biased closed position, in accordance with the present disclosure;

FIG. 6A shows the bottom perspective view of the movable jaw member of the electrode connector in accordance with the present disclosure;

FIG. 6B shows the bottom perspective view of the movable jaw member of the electrode connector in accordance with the present disclosure;

FIG. 6C shows the bottom perspective view of the main connector body of the electrode connector in accordance with the present disclosure;

FIG. 7A shows a cutaway sectional view illustrating electrical contact between the contact of the electrode connector and the stud of a biomedical patient electrode in accordance with the present disclosure;

FIG. 7B shows a cutaway sectional view illustrating electrical contact between the multiple contacts of the electrode connector and the stud of a biomedical patient electrode in accordance with the present disclosure;

### DETAILED DESCRIPTION OF THE DISCLOSURE

The present disclosure may be understood more readily by reference to the following detailed description taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Any and all patents and other publications identified in this specification are incorporated by reference as though fully set forth herein.

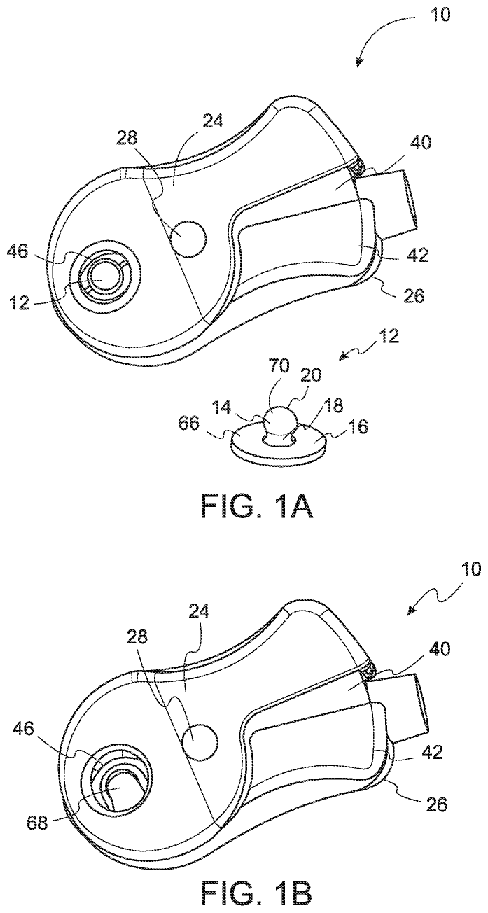

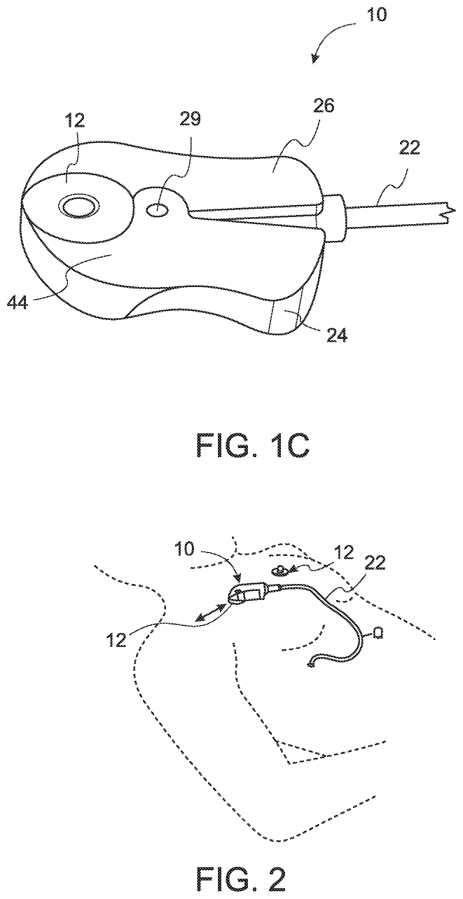

With reference now to the drawings, FIGS. 1 through 7 illustrate preferred and alternate embodiments of an electrode or electrical connector, generally referenced as 10, for use with a biomedical patient electrode or stud 12 of the type commonly used in EKG/ECG monitoring (hereinafter “ECG”). For ease of reference, FIGS. 1A, 1C, 2, 3A, 3C, 4A, 4C, 5A, 5C, 6A, 6C, and 7A refer generally to the preferred embodiment of an electrode connector comprising a single contact 64. FIGS. 1B, 1D, 2, 3B, 3C, 4B, 4D, 5B, 5D, 6B, 6C, and 7B refer generally to an alternative embodiment of an electrode connector comprising multiple contacts 64, 68. An additional alternative embodiment disclosed herein is an electrode connector comprising a second conducting contact 68 without the first conducting contact 64.

As illustrated in FIGS. 1A and 1B (top view) with stud 12, and FIG. 1B (bottom view), the present disclosure provides an improved, closed-end ECG electrode connector 10 which is specifically adapted for attachment to a biomedical patient electrode 12, as more fully described herein.

FIGS. 1A, 1B and 1C show a biomedical electrode 12 with an upwardly projecting electrode stud 14, which includes a radially enlarged electrode base 16, a stud neck 18, and a stud head 20. Electrode connector 10 is in electrical communication with an elongate flexible, electrically conducting leadwire 22 that extends therefrom and functions to transmit biomedical electrical signals to the patient monitoring system (not shown), as understood by one having ordinary skill in the art. The structure of the electrode connector 10 is alternatively fabricated from a radiolucent material, such as a suitable plastic, polymer or carbon, especially if the intent is for radiolucency.

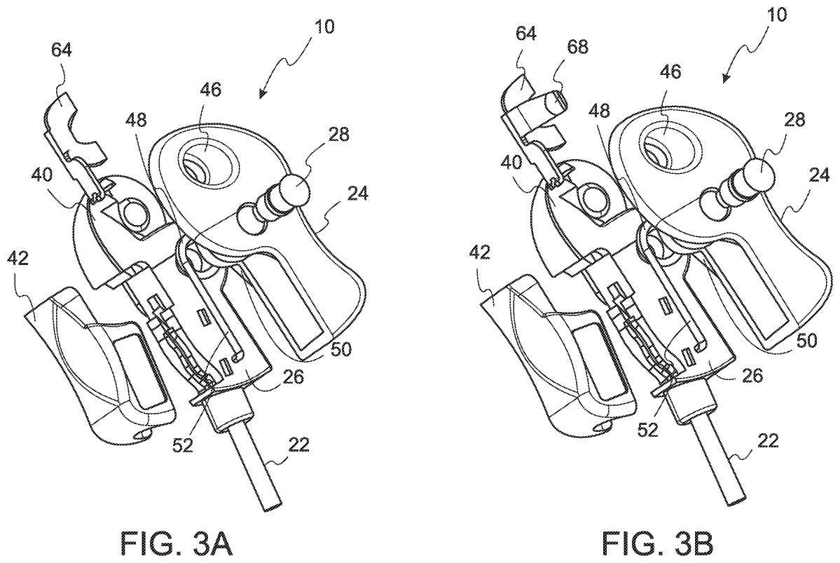

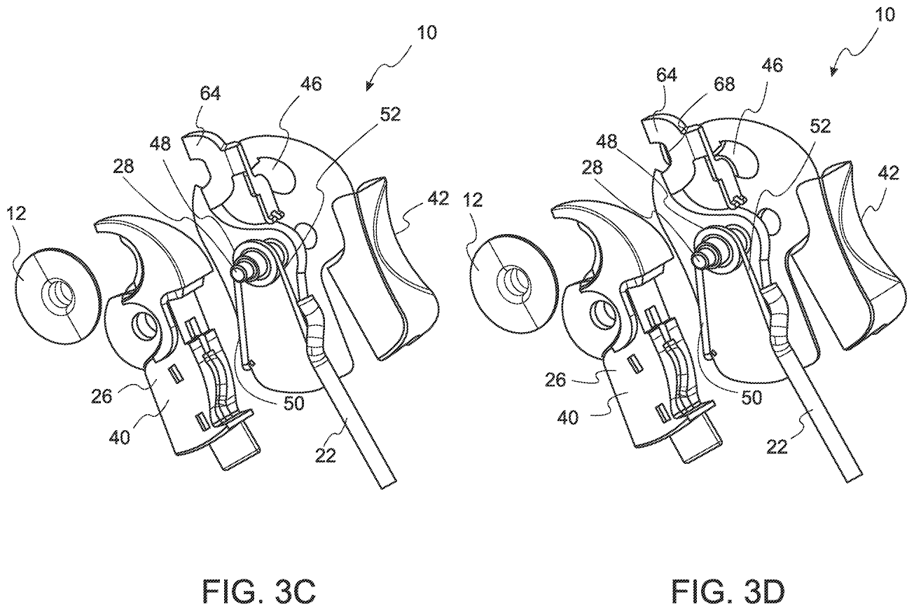

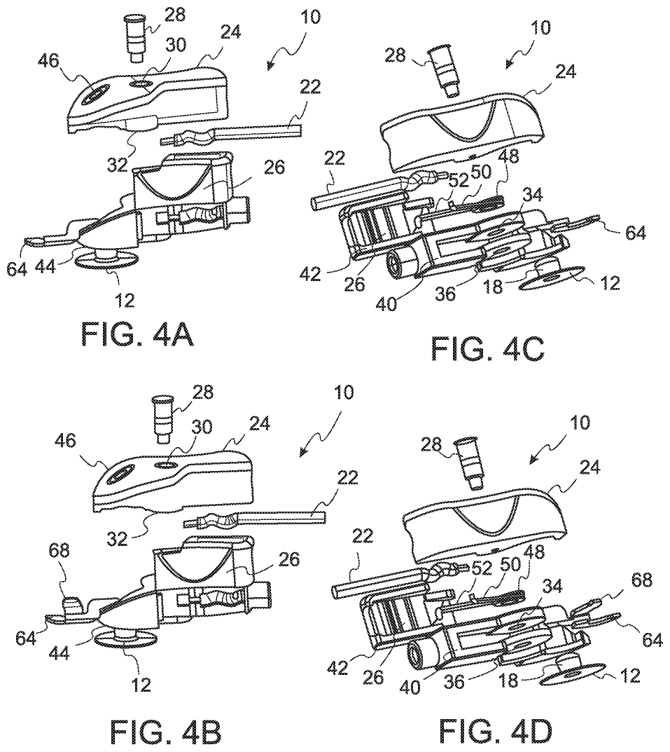

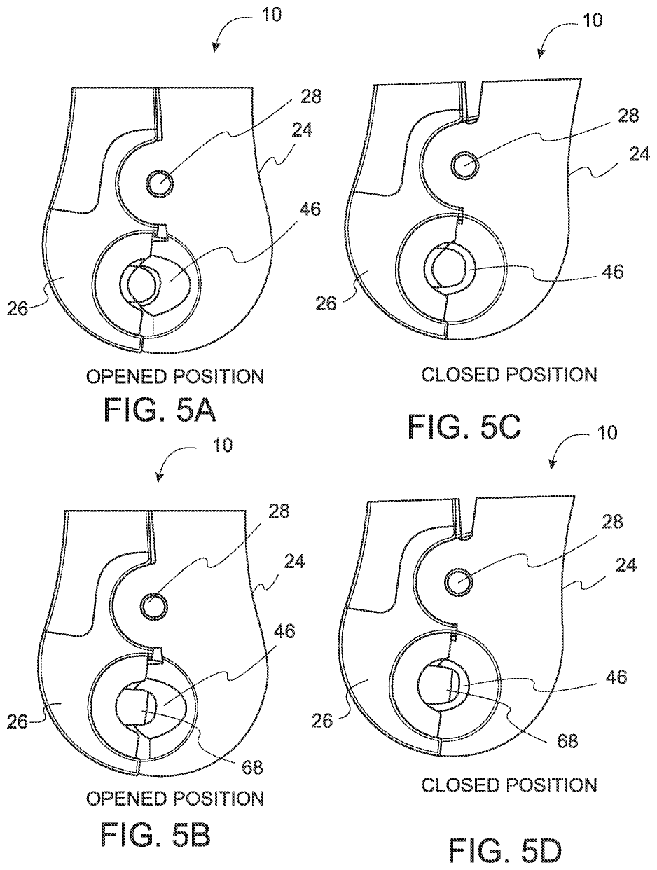

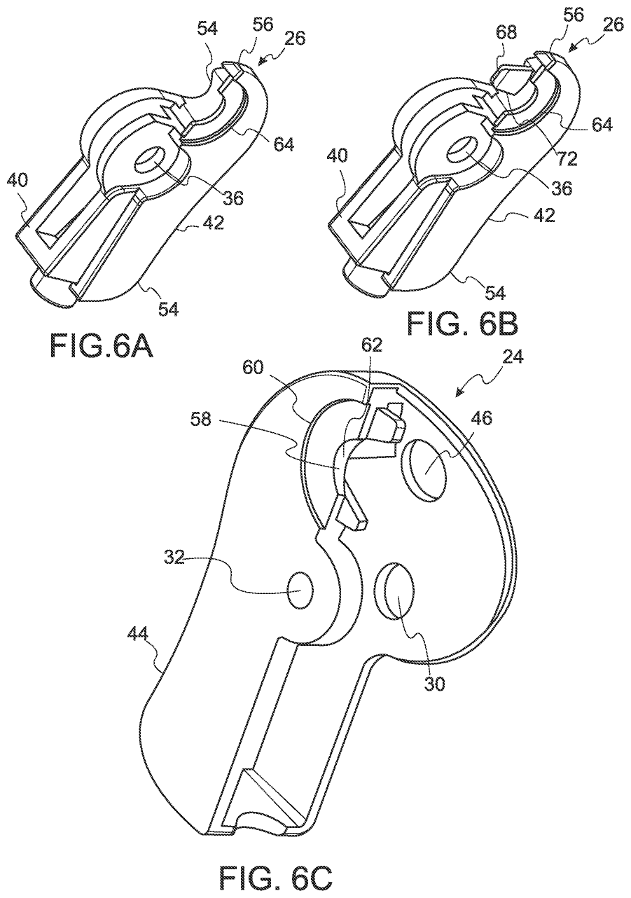

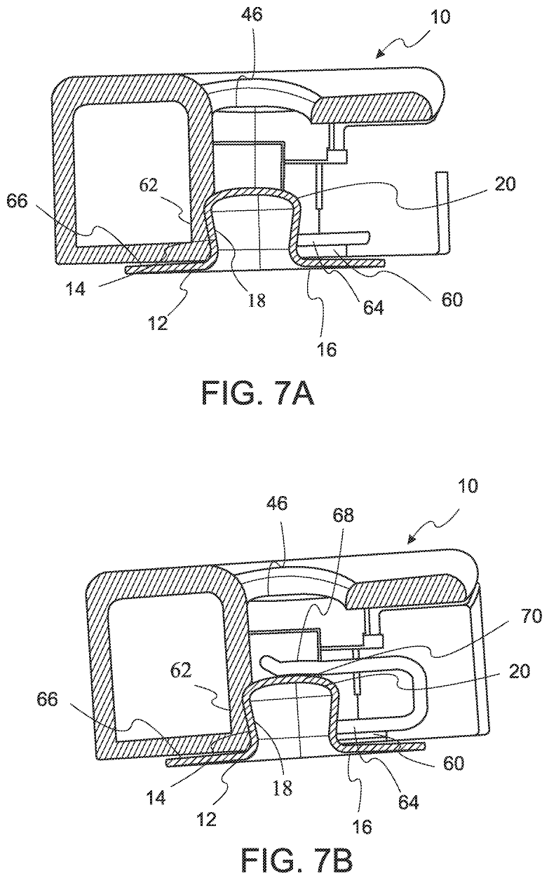

FIGS. 1 through 7 generally depict a preferred embodiment and alternative embodiments of an electrode connector 10, in accordance with the present disclosure. Electrode connector 10 preferably includes a main connector body 24, and a resiliently-biased, movable lever-actuated jaw member 26 pivotally connected thereto by a pivot connection 28, for example a pivot pin. FIGS. 1A-1C show top and bottom views of the electrode connector. FIGS. 3A-3D show exploded top and bottom views of the electrode connector. FIGS. 4A-4D show exploded side views of the electrode connector. FIGS. 5A-5D show top views of the electrode connector when in the opened and closed positions. FIGS. 6A-6C show perspective views of the jaw member and the connector body of the electrode connector. FIGS. 7A-7B show cutaway views of a closed electrode connector around a stud 12.

The main connector body 24 comprises a first main connector opening 30 and second main connector opening 32 configured to receive the pivot connection 28. Likewise, the jaw member 26 comprises a first jaw member opening 34 and a second jaw member opening 36 that are configured to interleave with the first and second main connector body openings 30, 32 and to receive the pivot connection 28, thereby connecting the main connector body 24 and the jaw member 26. The pivot connection 28 can be configured to pressure fit into the last opening at the bottom of the main connector body 24, or the pivot connection 28 can be configured to fit into a pivot connection end cap or a molded piece 29.

The main connector body 24 further comprises a clamping prong 62 configured and located to assist with optimizing electrical connectivity with the electrode stud 14. The retaining aperture can be different shapes, but in the preferred embodiment, the clamping prong 62 is arc shaped. In use, when the electrode connector 10 is in the opened position (FIGS. 5A and 5B), the electrode stud 14 is located in the opening 46. As the electrode connector 10 moves to the closed position (FIGS. 5C and 5D), the electrode stud 14 is pushed or forced by the clamping prong towards the first conducting contact 64, thereby holding the electrode stud 14 against the first conducting contact 64 and in the best position to make optimal electrical contact.

Movable jaw member 26 preferably comprises a jaw member main portion 40 and a jaw member lever portion 42. The jaw member main portion 40 and the jaw member lever portion 42 can be separate to simplify the assembly of the electrode connector 10, but may also be combined into an integral jaw member 26, depending on the design or functionality. For ease of reference, jaw member 26 will be described herein and will refer to both portions, jaw member main portion 40 and jaw member lever portion 42, unless reference to one or the other is specified.

As described herein, the movable jaw member 26 is biased to a closed position, such that if there is no force placed on the lever portion 42 of the jaw member 26, the electrode connector 10 will be biased to its closed configuration, as seen in FIGS. 1A, 1B, 1C, 5C, 5D, 7A and 7B. Main connector body 24 and jaw member 26 pivot relative to one another about pivot connector 28.

The present disclosure further comprises a bottom side 44 of the main connector body 24 having a closed-end defining an opening 46 which extends fully though the connector, namely from top to bottom. Opening 46 functions to receive the stud 14 of the biomedical patient electrode 12. Providing an electrode connector 10 with a closed-end avoids potential snagging of the one or more leadwires 22 thereby preventing inadvertent detachment of patient electrodes 12 and/or electrode connectors 10. Opening 46 can also function to ease installation of the electrode connector 10 by providing a line of sight (around an alternative second conducting contact 68, if present) through the electrode connector 10 to assist the user in aligning the electrode connector 10 with the biomedical electrode stud 14.

In a preferred embodiment, jaw member 26 is biased to the closed position in which the electrode connector 10 engages the stud 14, as described herein, and in which the jaw member 26 may be manually actuated to a disengaged or open position as illustrated in FIGS. 5A and 5B. FIGS. 3A-3D, and 4A-4D are exploded perspective views of an electrode connector 10 as disclosed herein.

A biasing member 48, such as a torsion spring, is contained within the electrode connector 10 to provide the biasing force for maintaining the electrode connector 10 in the closed configuration. Biasing member or spring 48 has opposing first leg 50 and second leg 52, that are connected to or bear against each of main body 24 and jaw member 26, respectively.

While a coil-type spring 48 is illustrated, the present disclosure contemplates any suitable biasing device 48. In an alternative embodiment wherein electrode connector 10 is configured to be radiolucent, spring 48 may be fabricated from a radiolucent material, such as silicone, rubber, plastic or other suitable radiolucent resilient material. More particularly, in a contemplated alternate embodiment, a resilient block of material, such as silicone or another suitable resilient radiolucent material, may be used in lieu of spring 48 to bias jaw member 26 to the closed position or configuration.

Jaw member 26 includes a rear portion 54 defining a lever actuating structure (which may be part of the separate lever 42) and a front portion 56 that forms a jaw defining an interior concave surface forming a concavity 58. The concave surface 58 functions to receive and engage the head 20 and/or neck 18 of an electrode stud 14.

As described above, the jaw member 26 further defines at least one, and preferably, a pair of aligned openings 34, 36 disposed between the respective front portion 56 and rear portion 54 for receiving pivot connection 28. Rear portion 54 defines an exterior concave surface and functions as a finger-receiving actuation structure whereby a user may manually manipulate the lever member 54 between closed configuration, illustrated in FIGS. 5C and 5D, and an open configuration as illustrated in FIGS. 5A and 5B, using a finger or thumb. Front portion 56 functions as a movable jaw and includes a concavity 58 that functions in the closed configuration to secure the stud 14 of a patient electrode 12 in mechanical and electrical engagement as more fully described herein.

Additionally, a recessed portion 60 terminates within concavity 58 at or near the clamping prong 62. Clamping prong 62 functions to engage the neck 18 of patient electrode 12 upon spring biased closure of jaw 26. Clamping prong 62 is positioned so as to engage and force the stud 14 toward the first conducting contact 64, as described above, such that face-to-face electrical contact is made between the first conducting contact 64 and the neck 18 of the patient electrode 12, thereby significantly increasing electrical surface area contact.

In an alternative embodiment, the present disclosure allows for a front portion 56 with a beveled surface that functions, upon engagement with the head 14 of a biomedical electrode 12 to urge jaw member 26 to the open configuration, thereby allowing electrode connector 10 to be affixed to a patient electrode 12 by a snap connection. Beveled surface terminates within concavity 58 at a radially projecting lip. The lip functions to engage the lower portion of head 20 upon spring biased closure of jaw 26. Lip can be positioned so as to engage the stud 14 at the lower portion of head 20 and thus force the stud 14 upward toward a first conducting contact 64 such that face-to-face electrical contact is made between the first conducting contact 64 and the patient electrode 12 thereby significantly increasing electrical surface area contact.

At about the same time that the first conducting contact 64 is making electrical contact with the patient electrode 12, in an alternative embodiment, the second conducting contact 68 is located and configured to engage and contact the top side 70 of the stud 14. As such, the bottom side 72 of the second conductive contact 68 will slide over and make electrical connection with the top side 70 of the stud 14. Finely tuned overlapping stud retaining features between the main connector body 24 and the contact area of the movable jaw member 26 are designed to prevent the stud 14 easily disengaging if there is incidental force on the pinch that causes the movable jaw member 26 to move (but not fully open).

As described herein, the main connector body 24 further comprises a clamping prong 62 configured and located to assist with optimizing electrical connectivity between the electrode stud 14 and the first conducting contact 64 and alternatively, the second conducting contacts 68. In the preferred embodiment, the clamping prong 62 is an arc shape. When the electrode connector 10 is in the open position, the electrode stud 14 can be inserted and is located in the electrode connector opening 46.

When the electrode connector 10 is closed, the electrode stud 14 is forced into a position where it is tightly held for optimal electrical contact between the electrode stud 14 and the first conducting contact 64, and in alternative embodiments, between the electrode stud 14 and the second conducting contacts 68. This configuration optimizes the electrical conductivity when the electrode connector 10 is in the closed position.

Both the first conducting contact 64 and the alternative second conducting contact 68 are disposed within the jaw member 26 of the electrode connector 10, and can be separate contact portions or can be a single contact with different contact ends. Either way, the first conducting contact 64 and the second conducting contact 68, when used, are both in electrical communication with leadwire 22, which enters the electrode connector through the movable jaw member 26.

The first and second conducting contacts 64, 68 are fabricated from electrically conductive material. The first conducting contact 64 terminates in an arc shape to make optimal electrical connection with the neck 18 of the stud 14. Alternative shapes for the clamping prong 62 and the first conducting contact 64, besides an arc, include V-shape, square, triangular and any other shape that will increase the engagement of the clamping prong 62 and the stud 14, on the one hand, and the electrical connection between the first conducting contact 64 and the neck 18 of the stud 14. The second conducting contact 68 is alternatively configured in a planar shape to make optimal electrical connection with the top of the head 20 of the stud 14.

An alternative embodiment of the present disclosure relates to an electrode connector 10 adapted to engage a biomedical patient electrode 12 by snap connection. In accordance with this aspect of the present disclosure, jaw member 26 includes a beveled lower surface along the peripheral edge of concavity 58 that functions, upon contact with the head 20 of stud 14, to force jaw member 26 open such that electrode connector 10 may be affixed to the electrode 12 by press connection wherein the user merely aligns stud 14 using the line of sight provided by connector opening 46 and merely presses electrode connector 10 downward whereby the beveled surface minimizes the force required to move jaw member 26 against the spring biasing force 48 so as to enable stud 14 to pass there over. Minimizing the required press-force is an important feature as it prevents patient discomfort.

As such, the present disclosure contemplates multiple methods of affixing the novel electrode connector 10 to a biomedical patient electrode 12. The first method, the pinch-clip method, utilizes the lever portion 42 of the jaw member 26. After applying the biomedical patient electrodes 12 to the human body, as shown in FIG. 2, the user can hold the electrode connector 10 by placing the thumb and index finger on the main connector body 24 and the lever portion 42 of the jaw member 26, such that the bottom side 44 of the main connector body is facing away from the user. The user than can align the electrode connector 10 with the electrode stud 14 by viewing the head 20 of the electrode stud 14 through the opening 46 in the main connector body 24.

Next, the user depresses the lever portion 42 of the movable jaw member 26 towards the main connector body 24 to overcome the biasing force created by the biasing member 48. Once that biasing force is overcome, the electrode connector 10 will move from the closed configuration, shown in FIGS. 1A, 1B, 1C, 5C, 5D, 7A and 7B, to the open configuration, shown in FIGS. 5A and 5B. Now, the user can place the opening 46 over the head 20 of the electrode stud 14 and release the lever portion 42 thereby reducing the force against the biasing member 48 and returning the electrode connector 10 back to its closed position or configuration, but this time with the first conducting contact 64 and the second conducting contact 68, if used, making optimal electrical contact with the stud neck 18 and the top side 70 of the electrode stud 14, respectively.

The alternative method for affixing the novel electrode connector 10 to a biomedical patient electrode 12, the snap or press method, utilizes the beveled surface of the main connector body 24. Again, the user holds the electrode connector 10 such that the bottom side 44 of the main connector body 24 is aligned with the electrode stud 14. The electrode connector 10 is then snapped or pressed onto the biomedical patient electrode 12 as described herein.

Next, the user presses the opening 46 over the head 20 of the electrode stud 14, such that the beveled surface will assist in overcoming the biasing force created by the biasing member 48, thereby urging the electrode connector 10 into the open position. Once that biasing force is similarly overcome, the opening 46 in the electrode connector 10 will allow the electrode stud 14 to enter the opening 46. The force against the biasing member 48 will then be reduced and the electrode connector 10 will return back to its closed position, again with the first conducting contact 64, and the second conducting contacts 68, if used, making optimal electrical contact with the stud neck 18 and the top side 70 of the electrode stud 14, respectively. Once completed, as shown in FIG. 2, the biomedical patient electrode 12 can be applied to the patient.

To remove the electrode connector 10 from the electrode stud 14, the user can depress the lever portion 42 of the movable jaw member 26 towards the main connector body 24 to overcome the biasing force created by the biasing member 48. Once that biasing force is overcome, the electrode connector 10 will again move from the closed configuration to the open configuration, and it can easily be removed from contact with the electrode stud 14.

As described herein, an alternative embodiment electrode connector 10 in accordance with the present disclosure includes the electrode connector 10 described above in which the electrode connector 10 comprises both a first conducting contact 64 and a second conducting contact 68. Another alternative embodiment electrode connector 10 in accordance with the present disclosure includes the electrode connector 10 described above in which the electrode connector 10 comprises only a second conducting contact 68.

The alternative embodiment of the present disclosure also includes the possibility of providing the front portion 56 with a beveled surface that functions, upon engagement with the head 14 of a biomedical electrode 12 to urge jaw member 26 to the open configuration, thereby allowing electrode connector 10 to be affixed to a patient electrode 12 by a snap connection. Again, beveled surface terminates within concavity 58 at a radially projecting lip. Lip functions to engage the lower portion of head 20 upon spring biased closure of jaw 26. Lip can be positioned so as to engage the stud 14 at the lower portion of head 20 and thus force the stud 14 upward toward a first conducting contact 64 thereby significantly increasing electrical surface area contact.

The first conducting contact 64 is disposed within the electrode connector 10, and is in electrical communication with leadwire 22. The first conducting contacts 64 preferably comprises an arc-shaped contact fabricated from electrically conductive material.

Another alternative embodiment of the present disclosure again includes the possibility of providing the front portion 56 with a beveled surface that functions, upon engagement with the head 14 of a biomedical electrode 12 to urge jaw member 26 to the open configuration, thereby allowing electrode connector 10 to be affixed to a patient electrode 12 by a pinch connection or a snap connection. The second alternative embodiment does not include a first conducting contact 64, but only comprises the second conducting contact 68, which is located and configured to engage and contact the top side 70 of the stud 14. As such, the bottom side 72 of the second conductive contact 68 will slide over and make electrical connection with the top side 70 of the stud 14.

The second conducting contact 68 is disposed within the electrode connector 10 and is in electrical communication with leadwire 22. The second conducting contact 68 preferably comprises a plate or plates fabricated from electrically conductive material. Additionally, the material used could include a compressible conductive material, such as conductive foam or rubberized material that provides a more expansive contact area with the stud 14.

In another alternative embodiment, the electrode connector 10 is made from radiolucent materials and the electrode connector 10 is intended to be radiolucent. The main connector body 24 and all of the components of the main connector body 24 can be manufactured from a radiolucent material. Additionally, the jaw member 26 and all of the components of the jaw member 26 can be manufactured from a radiolucent material. The first and second conducting contacts 64, 68 may be fabricated from radiolucent material, otherwise they may be fabricated from a suitable electrically conductive metallic material, such as steel or copper.

Another novel aspect of the present disclosure and yet another alternative embodiment involves providing an electrode connector 10 adapted from an improved electrical connection with the biomedical electrode 12. This aspect is most significant in embodiments wherein the entire electrode is fabricated from radiolucent materials, particularly including the first and second conducting contacts 64, 68 (or one or the other separately) that contacts the patient electrode 12 as electrical conductivity between radiolucent contacts 64, 68 and the patient electrode 12 is less than between a metal plate and a patient electrode 12. Accordingly, maximizing the surface contact area between the radiolucent contacts and the patient electrode 12 is critical to ensuring optimal signal detection and transmission.

The present disclosure has been shown and described herein in what is considered to be the most practical and preferred embodiment. It is recognized, however, that departures may be made therefrom within the scope of the disclosure and that obvious modifications will occur to a person skilled in the art.

As used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Along those lines, reference throughout the specification to “alternative embodiments,” “various embodiments,” “some embodiments,” “one embodiment,” “an embodiment”, or the like, means that a particular feature, structure, or characteristic described in connection with the embodiment is included in at least one embodiment. Thus, appearances of the phrases “in alternative embodiments,” “in various embodiments,” “in some embodiments,” “in one embodiment,” or “in an embodiment”, or the like, in places throughout the specification are not necessarily all referring to the same embodiment.

Further, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

Although numerous embodiments of this invention have been described above with a certain degree of particularity, those skilled in the art could make numerous alterations to the disclosed embodiments without departing from the spirit or scope of this disclosure.

All directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the any aspect of the disclosure.

As used herein, the phrased “configured to,” “configured for,” and similar phrases indicate that the subject device, apparatus, or system is designed and/or constructed (e.g., through appropriate hardware, software, and/or components) to fulfill one or more specific object purposes, not that the subject device, apparatus, or system is merely capable of performing the object purpose. Joinder references (e.g., attached, coupled, connected, and the like) are to be construed broadly and may include intermediate members between a connection of elements and relative movement between elements. Accordingly, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An electrode connector for establishing electrical connection to a biomedical patient electrode having an upwardly projecting electrode stud from a top surface thereof, the stud including a radially enlarged base, a stud neck, a stud head and a top of stud head, said connector comprising:
a main connector body comprising a retaining portion, and having a first main connector opening and a second main connector opening, said main connector body defining a closed ended structure with an opening extending through the main connector body;
a jaw member pivotally connected to said main connector body, said jaw member having a first jaw member opening and a second jaw member opening;
said jaw member further comprising a first electrically conducting contact and a second electrically conducting contact, said first electrically conducting contact disposed in electrically conducting contact with a portion of the stud upon attachment of said connector to the stud, said second electrically conducting contact disposed in electrically conducting contact with a portion of the stud upon attachment of said connector to the stud;
said main body and jaw member pivotably connected to each other through said first main connector opening and said second main connector opening and said first jaw member opening and said second jaw member, using a pivot connection;
said main body and jaw member configurable between an open configuration and a closed configuration;
a biasing member engaging said main connector body and said jaw member and biasing said members to the closed configuration.

2. The electrode connector according to claim 1, wherein said first conducting contact is an arc-shaped contact.

3. The electrode connector according to claim 2, wherein said arc-shaped contact is configured to make contact with said stud neck when said main body and jaw member change from an open configuration to a closed configuration.

4. The electrode connector according to claim 1, wherein said second conducting contact is a plane-shaped contact.

5. The electrode connector according to claim 4, wherein said plane-shaped contact is configured to make contact with said top of said stud head when said main body and jaw member change from an open configuration to a closed configuration.

6. The electrode connector according to claim 1, wherein said biasing member comprises a spring.

7. The electrode connector according to claim 1, wherein said biasing member comprises resilient material.

8. The electrode connector according to claim 7, wherein said resilient material is radiolucent.

9. The electrode connector according to claim 1, wherein said retaining aperture is an ovoid shape.
